(19) United States
(12) Patent Application Publication
(10) Pub. No.: US 2012/0070883 A1
(43) Pub. Date: Mar. 22, 2012

(54) HIGH TEMPERATURE HIGH PRESSURE MICROBIAL REACTOR

(76) Inventors: F. Prescott WARD, Indialantic, FL (US); Carsten Brehm, Carvel (CA); Markus Lemke, Edmonton (CA); Kurt Artur Semler, Edmonton (CA)

(21) Appl. No.: 13/110,758

(22) Filed: May 18, 2011

Related U.S. Application Data
(63) Continuation-in-part of application No. 12/884,693, filed on Sep. 17, 2010.

(51) Int. Cl. C12M 1/00

(52) U.S. Cl. 435/283.1

(57) ABSTRACT

The high temperature/high pressure (HTHP) reactor of the present invention includes a main cylinder, a reversible piston inside the cylinder that divides the main cylinder into a pressurization chamber and a reactor chamber, a thermal jacket, a lid and an end cap. The HTHP reactor of the present invention is configured to facilitate microbial growth in the reactor chamber that provides high temperature and high pressure conditions that simulate resource reservoir conditions, wherein the resource may be an oil reservoir. The HTHP chamber of the present invention is configured to receive a fluid sample from the underground oil reservoir that has been maintained at reservoir temperature and pressure throughout sampling, transfer to transport bottle, transport to the laboratory, and inoculation into the chamber. The HTHP chamber of the present invention is also configured to allow the use of a variety of instrumentation and valves that can be customized to allow a user to monitor desired physical properties within the reaction chamber and the microbial behavior and byproducts. This information is used to create an individual treatment plan for a reservoir to maximize the resource recovery, for example during Microbiobial Enhanced Oil Recovery (MEOR).

### CROSS-REFERENCE TO RELATED APPLICATIONS

This application claims the benefit of U.S. Provisional Patent Application No. 61/243,472 having a filing date of Sep. 17, 2009 and is a continuation-in-part of U.S. Non-Provisional patent application Ser. No. 12/884,693 having a filing date of Sep. 17, 2010. The disclosures and teachings of both related applications identified above are hereby incorporated by reference.

### BACKGROUND OF THE INVENTION

Approximately sixty-five percent of all the oil discovered remains trapped underground in reservoirs following primary production (natural reservoir pressure) and secondary production (water or gas flood). Microbial enhanced oil recovery (“MEOR”) holds considerable promise for recovering a significant proportion of trapped global oil reserves.

Conventional MEOR is an empirical process whereby inexpensive nutrients are pumped into an oil reservoir to stimulate growth of indigenous, dormant microorganisms. In theory, the rejuvenated microbial community produces environmentally friendly biometabolites such as gases, acids, solvents, and surfactants that release trapped oil and/or biomass and polymers that plug water channels thereby diverting subsequent water or gas floods into oil bearing zones.

Conventional MEOR has been employed for decades and has been moderately successful but, frequently, the results have been disappointing. A typical MEOR approach is to pump molasses and/or agricultural fertilizer into a watered-out reservoir and hope for the best. This hit-or-miss approach is not based on scientific principles and any positive, negative, or damaging results remain unexplained. In some cases, undesirable bio-metabolites such as hydrogen sulfide have caused irreversible reservoir damage, equipment corrosion, and health threats.

There are many applications of MEOR, but none of them include prior metabolic characterization of microbial communities that inhabit oil reservoirs. According to some culture-based and genetic evidence, microbial communities are markedly different among oil reservoirs depending on rock type, temperature, depth, and various other factors. Therefore, blindly injecting nutrients into an oil reservoir and hoping for beneficial results is an uncertain and potentially damaging process. Pumping the same nutrient into several reservoirs and expecting similar results is unscientific and unreasonable. There is no way currently to predict what bio-metabolic response, if any, can be expected in a given oil reservoir when nutrients are injected. Therefore, it would be beneficial to have a device for growing reservoir microorganisms in a controlled and scientific way to determine the optimal growth conditions and production of metabolic byproducts in a certain reservoir through laboratory experimentation that maintains and replicates the bottomhole temperatures and pressures.

Targeted, scientifically-based MEOR treatments could be devised for individual oil reservoirs if one knew the likely metabolic response of the microbial community to an infusion of nutrients. Then one could stimulate the desirable microbes and suppress the undesirable ones, for example, suppressing the sulfate-reducing bacteria responsible for souring oil. It is important to ascertain what the reactions the microbial community in a given reservoir has to nutrient infusions, what bioproducts they are capable of producing, and exactly what nutrients and co-factors are needed to grow at optimum rates. However, most reservoir microbes die when brought to the surface in a sampler, due to being exposed to air, low temperature, low pressure, and a variety of other stressors. Few, if any, indigenous microbial species survive when hoisted to the surface. Therefore, conventional laboratory culture of oil-reservoir microorganisms in Petri dishes or in flasks of liquid growth media at room temperature is not feasible. Some high temperature high pressure growth chambers have been attempted. Several of these attempts have required the introduction of an inert gas along with the sample to provide the proper pressurization. These methods and growth chambers do not replicate bottomhole conditions. It is also very difficult to simultaneously maintain an elevated pressure and temperature during the entire process of transferring the sample and adding an inert gas which results in losing a substantial portion of the viable material due to changes in temperature or pressure.

Therefore, it would be beneficial to have a microbial reactor that replicates and maintains the anaerobic, high temperature and pressure conditions of an underground reservoir in a laboratory setting without the addition of an inert gas. It is particularly desirable to substantially maintain the anaerobic, high temperature and pressure bottomhole conditions during the transfer of the down-hole fluid sample from the sampler or a transport vessel into a HTHP microbial reactor. It would further be beneficial to have a HTHP microbial reactor that facilitates growing the indigenous, dormant reservoir microorganisms of an underground reservoir under high temperature and pressure conditions while providing instrumentation to observe the results and bi-products of their growth when a variety of nutrients, stimulants or other conditions are present.

### SUMMARY OF THE INVENTION

The present invention is directed toward a high temperature/high pressure (HTHP) reactor that provides a growth chamber for microorganisms collected from or intended for introduction into HTHP (alternatively, HT or HP) environments. The present invention provides pressure and temperature continuity for bottomhole-sampled reservoir microbes that are transported to the laboratory, transferred to the HTHP growth chamber, and then grow and metabolize under conditions of high temperature and high pressure as if they never left the reservoir. This temperature and pressure continuity maintained in obtaining, transporting, inoculating, and studying reservoir microbial consortia in the laboratory is the key discriminator that separates this novel HTHP technology from all other current studies and applications of MEOR. The present invention is configured to be able to simulate reservoir and other HTHP environments in the laboratory, thus facilitating research, development, engineering, and other activities. The primary use of the HTHP reactor is to study growth, metabolism, and product formation of microbes under HTHP conditions, usually in a liquid environment. Other uses include studying biological, chemical, and/or physical interactions of microbes and substrates in HTHP environments, and studying, assessing, and/or evaluating other biological, physical, and/or chemical reactions and phenomena under HTHP conditions.

Potential uses of the HTHP reactor include but are not limited to culturing the following: a) oil and brine reservoir microorganisms ex situ (i.e. in the laboratory) in a variety of growth media under high-temperature high-pressure (HTHP) conditions that mimic reservoir conditions; b) microorganisms collected from other hydrocarbon formations including but not limited to heavy oil formations, oil sands (tar sands), tight oil and tight gas formations, coal seams, natural gas formations, oil shales (kerogen) and other intermediate stages of hydrocarbon formation, and deep-ocean gas hydrates (methyl clathrates); c) any and all extremophiles or facultative microorganisms from other HTHP environments including but not limited to those that colonize uranium, precious metals, and other subterranean ore deposits, deep ocean environments especially hydrothermal vents, salt domes and deposits, aquifers, and nuclear and other deep geological waste-disposal or waste-injection sites; and d) natural and engineered microbes destined for introduction into HTHP environments.

Environmental or experimental samples suitable for inoculation into the reactor chamber include not only fluid samples from subterranean reservoirs, ore bodies and other formations and sources, but also gases, slurries, emulsions, semi-solids, and/or solids especially if in a liquid milieu or if a suitable liquid milieu is added.

In addition, other items can be placed in the reaction chamber to study the various phenomena including (1) small sections of tubing, (2) wire, Teflon® or other mesh, (3) sandstone, carbonate, or other core or rock samples, and (4) other materials and substances that would provide substrates for attachment or otherwise elucidate formation of biofilms, metabolic activity, byproduct production and other phenomena.

The HTHP reactor of the present invention will have direct applications to studying the following: growth and metabolism of microorganisms and the formation of bioproducts involved in Microbial Enhanced Oil Recovery (MEOR); Carbon Capture and Sequestration (CCS) including biomineralization of injected CO₂; methanogenesis of hydrocarbons and other substrates; introduction of genetically engineered microbes into subterranean reservoirs for alkane and other hydrocarbon production; bioleaching of uranium, precious metals, and other ores; subterranean upgrading of oil sands, heavy oils, and other hydrocarbons by microbial, chemical, and/or physical means; effects of nutrient infusions into subterranean reservoirs; effects of chemical and physical treatments in HTHP environments, e.g., heat and energy treatments of subterranean and mined kerogen deposits; waste disposal in HTHP environments; various methods for bioreclamation and bioremediation under HTHP conditions; basic and applied studies on microbes from or to be introduced into HTHP environments; and other physical and chemical reactions, effects, and consequences under HTHP conditions.

In general, the HTHP reactor of the present invention includes a main cylinder, and a reversible piston within the main cylinder. The piston separates the interior of the cylinder into two distinct chambers, a pressurization chamber and a reaction chamber. The pressurization chamber is configured to receive the mechanism or method of adjusting the position of the piston Within the cylinder to increase or decrease the pressure within the reaction chamber. The reaction chamber is where the experiments on the growth metabolism and formation of byproducts by microorganisms take place. The HTHP reactor of the present invention is configured such that the piston maintains the bottomhole pressure on the reaction chamber such that no introduction of a foreign inert gas into the reaction chamber is necessary to pressurize the sample. The HTHP reactor of the present invention further includes a thermal jacket that is positioned over at least a portion of the main cylinder corresponding to the reaction chamber. The thermal jacket is configured to regulate and vary the temperature of the chamber to replicate the sample's in situ conditions. One embodiment includes a thermal jacket 16 that allows the passage of a heating or cooling fluid around the outside of the main cylinder. Alternatively, the thermal jacket may include electric heating elements to adjust and maintain the temperature of the reaction chamber.

The HTHP reactor includes a lid that is generally coupled to an end of the main cylinder. The HTHP lid also is configured to be coupled to the vessel jacket and may be in fluid communication with the vessel jacket allowing heating and/or cooling fluid to pass through the lid. The lid is generally configured such that a plurality of instruments can be mounted thereon. The instruments are generally configured to be in communication with the reaction chamber and measure various physical and chemical properties within the reaction chamber. The instruments assist the technicians in monitoring the growth and metabolism of the microorganisms, observe the byproducts made by the microorganisms, and/or provide a means to stimulate the contents in the reaction chamber. One embodiment of the present invention may include one or more of the following instruments: a pH indicator, a thermowell, a thermometer, a pressure gauge, a stirrer, and inlet or outlet valves to introduce or remove agents or samples. It is important to note that the present invention is configured to include active pH monitoring of the reaction chamber during the high temperature high pressure testing that, until now, was not possible in the current state of the art. Any instrumentation known or hereafter developed that would be useful in the experimentation may be mounted to the lid or reactor of the present invention and is within the scope of the present invention.

Further, the HTHP reactor of the present invention includes a closed end opposite the lid. Another embodiment includes an end cap that is coupled to the main cylinder at the end opposite the lid. The end cap may be configured to receive the connection for a pressurization system which may include a hydraulic or air hose, controls for a solenoid motor or other motor or other pressurization system or method known or hereafter developed. An alternative embodiment may include a vessel bottom member configured to be held in place against the end of the cylinder and to receive the pressurization input described above. In this embodiment, the end cap secures the vessel bottom to the main cylinder to seal off the end of the main cylinder. Yet another embodiment includes a closed end opposite the lid wherein the closed end results from welding a plate or cap over the end or machining the entire cylinder from a single piece of solid bar stock.

Other and further objects of the invention, together with the features of novelty appurtenant thereto, will appear in the course of the following description.

### BRIEF DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a cut-away perspective view of one embodiment of the HTHP reactor in fluid communication with a transport vessel in accordance with the teachings of one embodiment of the present invention;

FIG. 2 is a perspective view of one embodiment of the HTHP reactor in accordance with the teachings of one embodiment of the present invention;

FIG. 3 is a cross-sectional view cut along the line 3-3 of the embodiment of the HTHP reactor in accordance with the teachings of the embodiment of the present invention shown in FIG. 2;

FIG. 4 is a perspective view of one embodiment of the main cylinder of the HTHP reactor in accordance with the teachings of the present invention;

FIG. 5 is a cross-sectional view of one embodiment of the thermal jacket of the HTHP reactor cut along line 3-3 in accordance with the teachings of the embodiment of the present invention shown in FIG. 2;

FIG. 6A is a cross-sectional view of one embodiment of the lid of the HTHP reactor cut along line 3-3 in accordance with the teachings of the embodiment of the present invention shown in FIG. 2;

FIG. 6B is a cross-sectional view of another embodiment of the lid of the HTHP reactor in accordance with the teachings of the present invention;

FIG. 7A is a top view of the lid of one embodiment of the HTHP reactor in accordance with the teachings of one embodiment of the present invention;

FIG. 7B is a side view of one embodiment of the HTHP reactor in accordance with the teachings of one embodiment of the present invention; and

FIG. 8 is a cross-sectional view of one embodiment of the end cap of the HTHP reactor cut along line 3-3 in accordance with the teachings of the embodiment of the present invention shown in FIG. 3.

### DETAILED DESCRIPTION OF THE INVENTION

The following detailed description of the present invention references the accompanying drawing figures that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The present invention is defined by the appended claims and the description is, therefore, not to be taken in a limiting sense and shall not limit the scope of equivalents to which such claims are entitled.

One novel aspect of the HTHP reactor of the present invention is best understood in view of the circumstances surrounding its use. Therefore, a description how the HTHP reactor of the present invention is used is provided with the detailed description of the HTHP reactor. Once a bottomhole reservoir sample is collected and moved to the HTHP reactor, the HTHP reactor of the present invention substantially replicates the bottomhole temperatures and pressures allowing technicians to experiment with nutrient media formulations and concentrations that will provide for optimum microbial growth, maximum production of desirable byproducts, and suppression of undesirable microbes and byproducts. The sampling, transport, inoculation, and incubation phases are performed such that the HTHP chain is not broken. It is important that these conditions are not broken because a substantial amount of the native microbes will be killed. Based on these results and a review of the geology of the reservoir, a precise nutrient-medium formulation will be devised and injected into the reservoir in optimum quantities and will be allowed to remain for an optimum time to (a) plug watered-out channels, (b) to induce maximum production of other desirable biometabolites, and/or (c) to suppress growth and metabolism of undesirable microbes, especially those that produce H₂S and sour oil. The result is enhanced recovery of oil. The objective of this MEOR technology is to increase the amount of oil ultimately produced over what would have been recovered using other treatments.

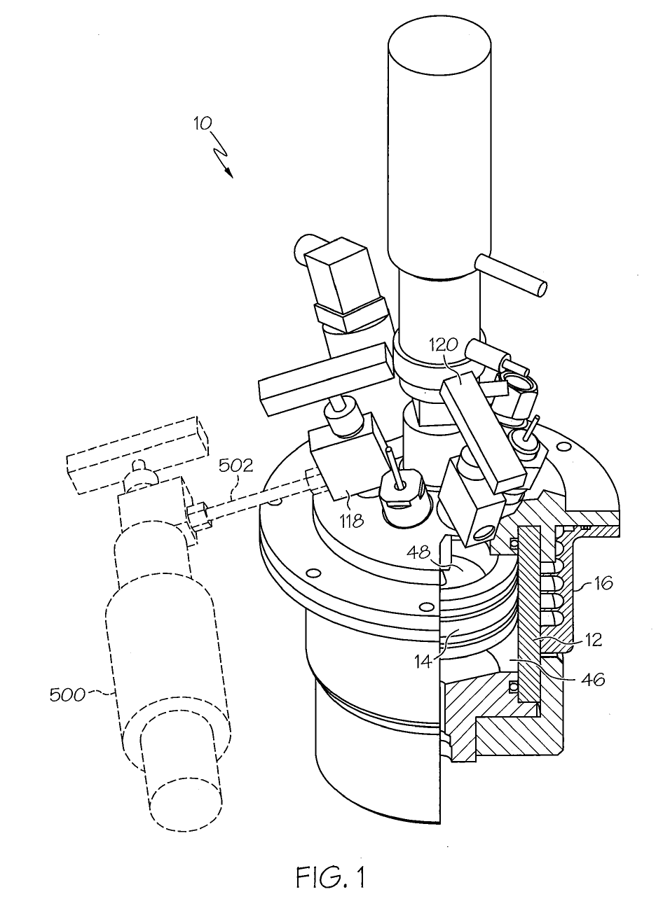

Using HTHP reactor 10 includes assembling HTHP reactor 10 as described below. An assembled HTHP reactor 10 is illustrated in FIG. 1. FIG. 1 further shows a transfer vessel 500 being in fluid communication with one embodiment of HTHP reactor 10 via tube 502.

Once a target reservoir is selected, bottomhole samples are obtained and the samples are maintained at reservoir temperatures and pressures during sampling, during transport to the laboratory and, using the HTHP reactor of the present invention, during culture in a laboratory. A conventional industry PVT-type sampler may be used to obtain bottomhole samples, usually bringing two each 600-ml fluid samples to the surface. The reservoir samples are usually obtained from the oil/water interface (residual oil zone), but can be harvested from any location within the reservoir. Additional samples can be obtained if required. Bottomhole temperature and pressure are measured by the sampler itself or by a separate probe and recorded while the sample is harvested. Pressure in the sampler's canisters is maintained at bottomhole pressure by hydraulics or nitrogen injection as canisters are retrieved to the surface. In some instances, the samples may be transferred into a transport vessel 500 by the sampling technician or contractor. The high temperature and pressure of the bottomhole environment is maintained through this transfer process such that the sample will continuously be subject to substantially the same temperature and pressure from the instant the sample has been taken though testing the sample in a laboratory using the HTHP reactor of the present invention. Prior to transfer to one or more HTHP reactors 10 of the present invention, reservoir samples contained in the sampler or transfer vessel 500 may be stored in an oven to maintain reservoir temperature. Reservoir bottomhole temperature and pressure are maintained inside the sampler and, if applicable, transfer vessels 500 throughout the entire sampling and transport processes. The chamber of the sampler or transfer vessel that contains the samples are not opened prior to fluid transfer because exposure to oxygen, low temperatures, and low pressures kills virtually all of the microbes.

In one embodiment, a reaction chamber 48 (as described below) of HTHP reactor 10 is loaded with selected sterilized nutrient media at ambient temperature and pressure. Growth and metabolic byproduct studies are often conducted of bottomhole microbial consortia in various liquid growth media including molasses, nitrogen/phosphate fertilizers, and various treatment grades of industrial wastes such as paper/pulp, sugar beet, brewery, feedlot, and municipal sewage that has undergone primary treatment. Further, many types of growth media are suitable for use including those typically used in empirical MEOR applications in the field. Some conventional MEOR solutions include but are not limited to: molasses (an inexpensive carbon source with micronutrients that is commonly used in MEOR), 0.5% aqueous solution (vol/vol) more or less; augmented molasses: 0.5% molasses, 0.15% KNO₃ (w/v), and 0.05% Na₃PO₄ (wlv), or variations thereof; or an aqueous solution of fertilizer: 0.25% KNO₃ (w/v), and 0.05% NaH₂PO₄ (w/v), or variations thereof.

One method of using the present invention (shown in FIG. 1) includes introducing about ninety (90) milliliters of sterile MEOR nutrient solution and/or industrial waste stream into reaction chamber 48 of one or more HTHP reactors 10 under ambient pressure through inlet valve 118. The pressurization system including fluid and/or gas introduced into pressurization chamber 46 (as described below) of HTHP reactor 10 such that piston 14 (as described below) compresses the nutrient solution or waste material to a pressure that substantially matches the measured bottomhole pressure of the sample. After reaction chamber 48 is loaded with nutrient media, reaction chamber 48 is pressurized using the pressurization system to the measured bottomhole pressure and the temperature of reaction chamber 48 is brought up to substantially match the recorded bottomhole conditions by heating reaction chamber 48 with thermal jacket 16 (as described below). The temperature and pressure can be regulated manually or using any control system now known or hereafter developed.

Only after the nutrient solution or industrial waste stream are brought to the bottomhole temperature and pressure, are about ten (10) milliliters of reservoir fluids from a single well added to reaction chamber 48 from transport vessel 500 at the bottomhole reservoir temperature and pressure, i.e. about a ten percent (10%) inoculum through tube 502. Larger or smaller volume HTHP reaction chambers can be used and inoculum ratios can be modified depending on requirements and growth responses. In addition, volumes of the above components may be increased or decreased from those disclosed herein. Any variations in the volume and percentage of nutrient media or industrial waste streams and inoculum are within the scope of the present invention.

A tubular connection 502 with a pressure gauge enables transfer of a portion of reservoir fluid (inoculum) from the sampler or transport vessel 500 to the loaded reaction chamber 48 of the present invention through inlet valve 118. In one embodiment, inlet valve 118 is opened to pressurize tube 502 and allow the nutrient in the reaction chamber that is at the bottomhole temperature and pressure to fill tube 502. Thus, when the sample is introduced into tube 502, it is already full of nutrient substantially at the bottomhole temperature and pressure. Thus, there is no discontinuity in temperature or pressure when transferring the sample from the sampler or transport vessel 500. The floating piston 14 allows for the nutrient to be introduced into the tube 502 and allows the sample to be pulled into reaction chamber 48 using differential pressures, but while preventing sudden pressure losses that result in killing the microbes in the reservoir fluid. During transfer of the sample of the reservoir fluid, the pressure in reaction chamber 48 is maintained at a pressure that is slightly less than the reservoir sample transport vessel 500 to provide for metered fluid flow into reaction chamber 48. The slightly less pressure is close enough to the actual bottomhole conditions that it does not have an adverse effect upon the sample. The position of the floating piston in main cylinder 12 may be gradually adjusted manually or through a control system to allow for a uniform pressure to be maintained in reaction chamber 48 even though the volume of liquid is increasing.

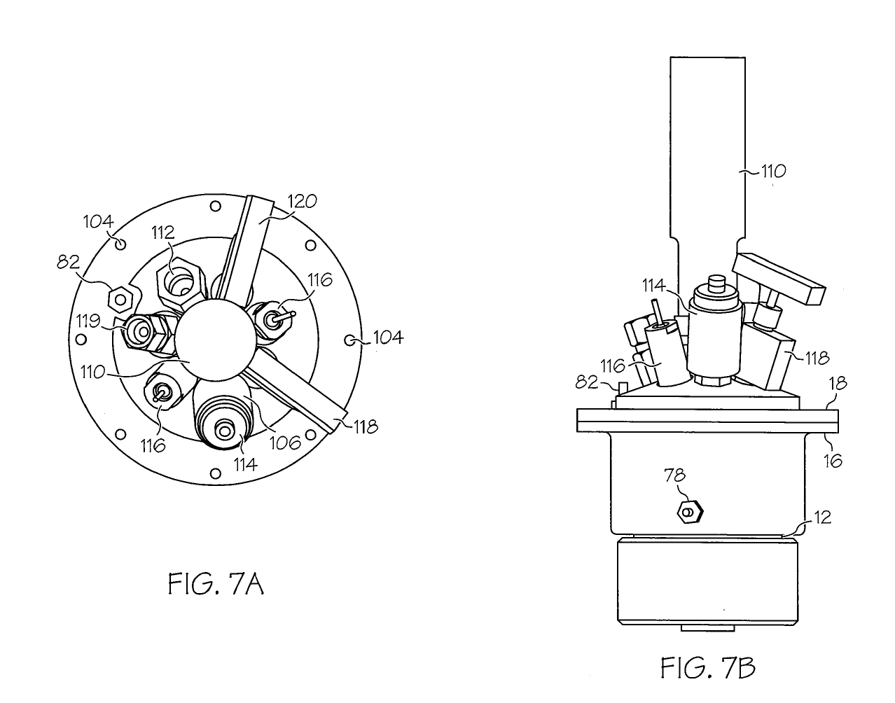

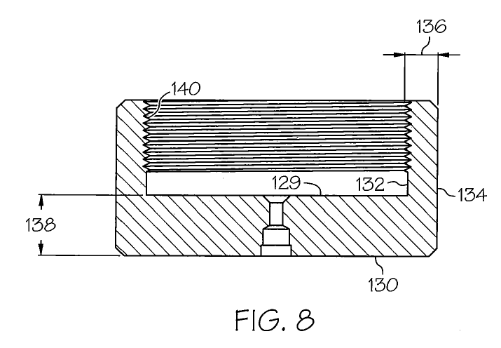

Once the inoculum has been introduced into reaction chamber 48, the pressure and temperature are monitored using thermowell 112 and pressure gauge 114 (both shown in FIG. 7A) and the pressure and temperature are kept at substantially identical conditions to the recorded bottomhole pressure and temperature using any control system now known or hereafter developed.

The growth of microbial consortia of various types may be assessed in the various dilutions of growth media by measuring (1) change in turbidity of growth medium, (2) numbers of microbes per ml (i.e., biomass), (3) volume of headspace gases produced, and (4) other measures of growth now known or hereafter developed. Other assessments may be performed and are within the scope of the present invention. Samples of headspace gases and liquid culture medium may be obtained out of outlet valve 120 for (1) chemical and volumetric analyses of headspace gases and (2) chemical nature of metabolic byproducts in the growth medium from microbial growth such as pH change, surfactants produced, polymers produced, and solvents produced.

By measuring biomass and by chemically analyzing bio-metabolites produced in the laboratory, one obtains accurate data to guide nutrient selection for a targeted reservoir, thereby insuring maximum release of trapped oil and mitigating risk of reservoir damage. Under HTHP culture in the HTHP reactor 10 of the present invention, the byproducts of microbes from a specific oil reservoir could be identified and predictions of growth and metabolism of the microbial consortium in the presence of a given nutrient mix could be obtained. By culturing the consortium in a number of nutrient growth media and chemically and physically measuring acids, gases, solvents, surfactants, biomass, and polymers produced, predictions could be made about specific metabolic byproducts to be expected in a given oil reservoir when injected with a specific nutrient medium at a given optimum formulation and concentration, and for a given optimum time for the injected well system to be shut in for the maximum MEOR effect. The optimum time can be determined by analyzing the metabolism rates for the concentration of nutrient medium or other method as now known or hereafter developed.

Measurements of acid, gas, and biomass production may be obtained in real-time using the instrumentation described below. Typical incubations are expected to take approximately two to six weeks each, and the end point is generally determined by cessation of acid and gas production. The volume and composition of metabolic off gases and pH of the nutrient medium may be analyzed in real-time or periodically in samples removed from the growth chamber to obtain gas-generation (via gas chromatograph) and acid-generation (via pH meter) curves for each reservoir-nutrient combination. Instrumentation is generally incorporated into the HTHP reactor of the present invention to monitor one or more of pH, pressure, temperature, gases, and other parameters and constituents remotely and in real time. Biomass is calculated during and at the end of incubation by cell count, turbidity, filtering and weighing, and/or other measurements to obtain microbial growth curves.

Following incubation, liquid samples are transferred to a chemical laboratory for analysis. HTHP reactor 10 may be cleaned and sterilized using acceptable methods. HTHP reactor 10 may also be disassembled, cleaned with a solvent to remove hydrocarbon residues, and then autoclave-sterilized at 121° C. or equivalent to prepare for re-use.

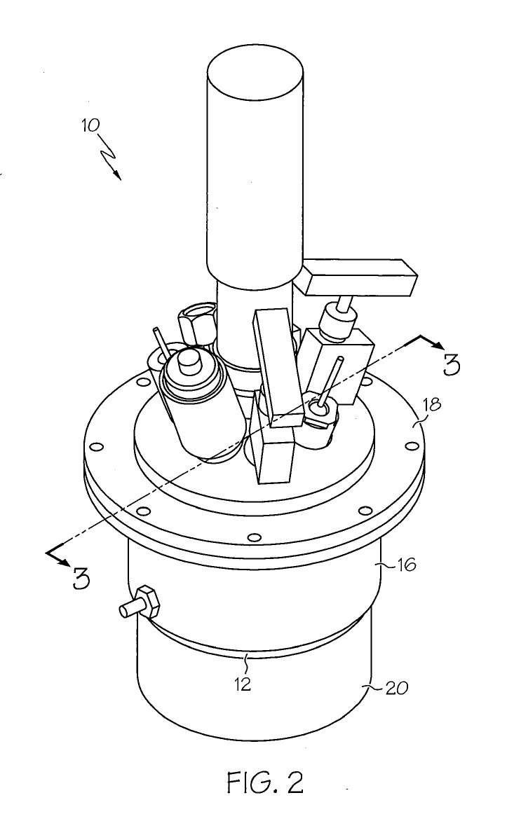

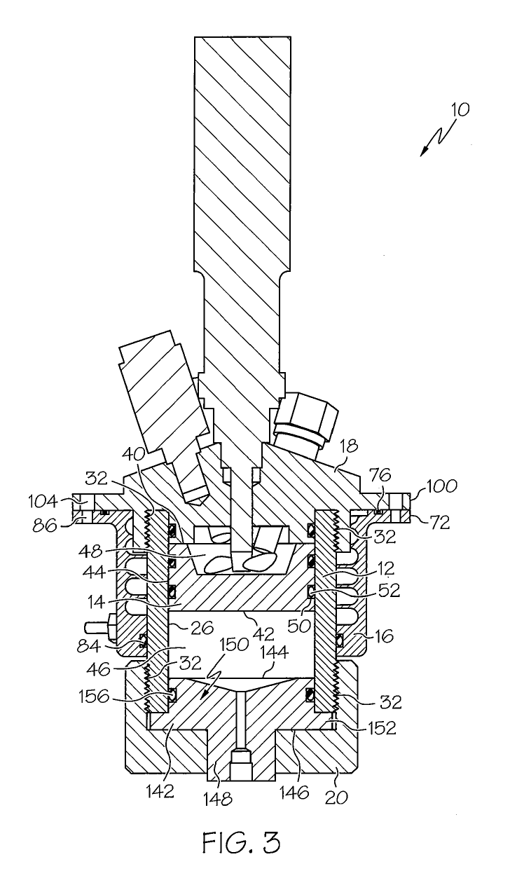

Now turning to FIGS. 2 and 3, the high temperature high pressure (HTHP) reactor 10 of the present invention generally comprises a main cylinder 12, a piston 14 inside main cylinder 12, a thermal jacket 16, a lid 18, and an end cap 20. The components of HTHP reactor 10 are generally configured to provide a ex situ testing chamber which replicates the temperature and pressure of in situ underground environments including, but not limited to, subterranean stores of natural resources such as oil, natural gas, and precious metals, or other commercially valuable ores or metals. The testing chamber will generally be used to grow and observe the products of microorganisms collected from or intended to be introduced into the underground environments using a variety of stimuli, food sources, and other environmental conditions.

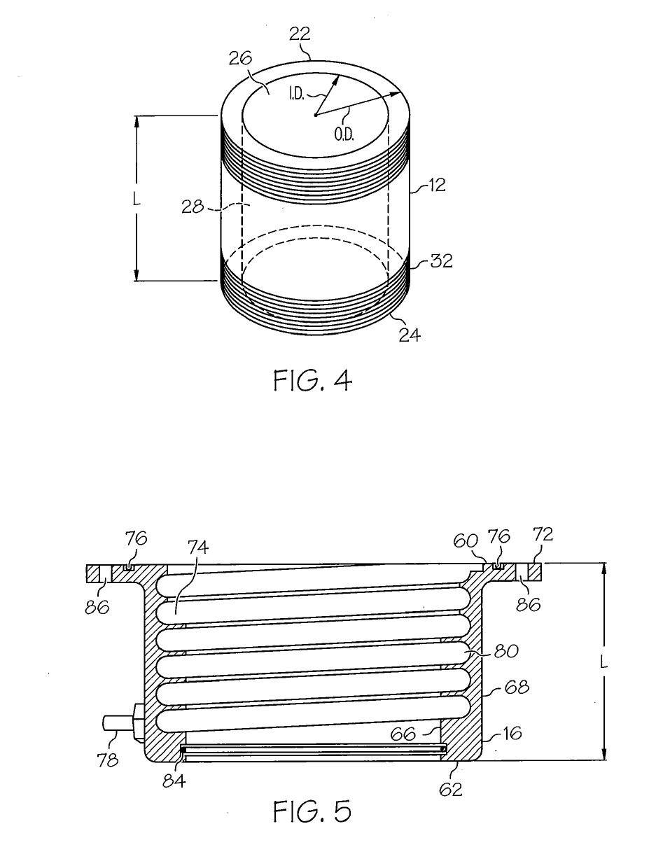

As shown in FIG. 4, main cylinder 12 includes a first end 22, a second end 24, an inner face 26, an outer face 28, a length defined by the distance between first end 22 and second end 24, and a wall thickness bounded by inner face 26 and outer face 28. Embodiments of main cylinder 12 may have any cross-section now known or hereafter developed including: rectangular, oval, circular, polygonal, triangular, or any other cross-section. One embodiment includes a circular cross-section. A circular cross-section lends itself to a high pressure chamber as the forces applied on the cylinder due to the differential pressure are evenly distributed throughout the entire material. Cross-sections that include planar faces intersecting at a corner generally have stress concentrations at the corners thereby resulting in locations that are prone to material failure. However, triangular, rectangular or other polygonal shapes may be used if the cylinder and joints (if any) are designed to resist the desired maximum pressure. Further, the HTHP reactor of the present invention may include a plurality of main cylinders 12 of a variety of lengths that can be used interchangeably depending on the volume of samples tested, the pressure required, or any other variable.

As further shown in FIGS. 3 and 4, first end 22 and second end 24 of main cylinder 12 are generally configured to be removably coupled to at least lid 18 and end cap 20. This embodiment further includes a portion of first end 22 and second end 24 having threads 32 wherein the ends 22, 24 are configured to threadably couple with lid 18 and end cap 20. Alternatively, one or more of first end 22, second end 24, lid 18, and end cap 20 may be flanged to facilitate a compression coupling using clamps, tie-down bolts, or other compression fitting now known or hereafter developed to couple the members together and providing pressure resistance.

An alternative embodiment not shown includes second end 24 of main cylinder being closed. The closed second end 24 may be machined through milling solid bar stock, or may include an end plate or cap seal welded to second end 24 of main cylinder 12, or any other method known or hereafter developed for producing a pressure resistant closed cylinder end. This alternative embodiment may further include a portion of the closed second end 24 of the main cylinder 12 being configured to allow second end 24 to receive, or be removably coupled to, an element of the pressurization system, including an air hose, a hydraulic hose, or other known components that are used to connect the pressurization system to HTHP reactor 10 for pressurizing the contents of pressurization chamber 46 thereby compressing pistion 14 against the contents of reaction chamber 48.

Piston 14 generally is housed inside cylinder 12 as shown in FIG. 3. Piston 14 includes a top face 40, a bottom face 42, and an outer surface 46. Piston 14 includes a depth defined between top face 40 and bottom face 42. In the embodiment shown in FIG. 3, piston 14 is circular. However, piston 14 may be any shape known and will generally correspond with the cross-section of main cylinder 12. As shown, piston 14 has an outer surface 44 corresponding to an outer diameter as shown in FIG. 3. The outer diameter of outer surface 44 of piston 14 is generally slightly less than the inner diameter of main cylinder 12 and piston 14 may travel linearly within main cylinder 12. The depth of piston 14 is generally less than the length of main cylinder 12 and the dimensions are configured to create a pressurization chamber 46 and reaction chamber 48 within main cylinder 12. In addition, top face 40 of piston 14 may be concave in profile as shown in FIG. 3 to further define the extents of reaction chamber 48. The volume of chambers 46 and 48 will vary depending upon the position of piston 14 within cylinder 12. Piston 14 is reversible meaning its position within cylinder 12 may actively be controlled in two directions to either increase or decrease the pressure within reaction chamber 48 to actively maintain the desired pressure in reaction chamber 48.

As further shown in FIG. 3, outer surface 44 may also include at least one notched channel 50 around its entire perimeter wherein notched channel 50 is configured is receive a seal member 52 that prevents fluid and gas from migrating between pressurization chamber 46 and reaction chamber 48. By preventing migration of fluid or gas between two chambers 46 and 48, one or more seal members 52 thereby allows pressure to be increased or decreased in reaction chamber 48 using increasing the volume of air in pressurization chamber 46 to force the floating piston 14 to apply pressure to the contents of reaction chamber 48. The pressure applied to reaction chamber 48 may be adjusted by increasing or decreasing the volume of gas in pressurization chamber 46 to increase or decrease the pressure of reaction chamber 48. Generally, HTHP reactor 10 of the present invention is configured with a pressurization system, including the pressurization chamber 46, which contains a combination of fluid and gas in pressurization chamber 46, a gas or fluid pump or compressor (not shown) that can apply pressure the sample in reaction chamber 48 by compressing piston 14 against the sample. HTHP reactor 10 of the present invention is configured to operate under and maintain an applied pressure on a sample in a range from about one-half mean sea level pressure (8 psi) to about ten-thousand pounds per square inch (10,000 psi). Alternatively, the pressure of reaction chamber 48 may be maintained by using a hydraulic or electric cylinder (not shown) to control floating piston 14 or a screw mechanism (not shown)

Thermal jacket 16 of HTHP reactor 10 generally facilitates adjusting the temperature of reaction chamber 48. In one embodiment of the present invention, thermal jacket 16 is capable of reaching and maintaining a temperature in reaction chamber 48 in a range of about zero degrees Celsius (0° C.) to about one-hundred degrees Celsius (100° C.). As best seen in FIG. 5, one embodiment of thermal jacket 16 generally includes a first end 60 and a second end 62 defining a length therebetween. Thermal jacket 16 generally includes an inner face 66, and an outer face 68 that defines a wall thickness between the two. Inner face 66 generally has a similar cross-section as outer surface 28 of main cylinder 12. When inner face 66 has a circular shape, as shown in FIG. 2, inner face is defined by an inner radius. As seen in FIG. 3, the inner radius of inner face 66 is slightly larger or the same as the outer radius of outer face 28 of main cylinder 12 thereby allowing thermal jacket 16 to slide over main cylinder 12 as shown in FIG. 3. Further, thermal jacket 16 may also include flange 72 wherein an embodiment of flange 72 includes a step 74 around the perimeter of inner face 66 proximate cylinder 12 as shown in FIG. 5. In addition, another embodiment of thermal jacket 16 may include a continuous notch in flange 72 that houses flange seal 76.

The embodiment of thermal jacket 16 shown in FIGS. 3 and 5 includes a liquid coolant flowing through thermal jacket 16. Thermal jacket 16 includes a coolant inlet 78 proximate second end 62 and includes a continuous spiral coolant channel 80 in inner face 66 along at least a portion of the length of thermal jacket 16 as shown. Coolant channel 80 is configured to allow coolant to flow from inlet 78 to an outlet. One embodiment includes a coolant outlet proximate first end 60. An alternative embodiment shown includes coolant outlet 82 on lid 18 as shown in FIG. 7A. The embodiment shown includes the interface between thermal jacket 16 and lid 18 allowing fluid communication between the two members. Heated or cooled fluid can pass through channel 80 and contact outer face 28 thereby transferring heat to or away from reactor chamber such that the temperature of reaction chamber 48 may be set and maintained to substantially mimic the thermal conditions of the downhole reservoir.

For the most efficient transfer of heat through the cooling channels, the interface between main cylinder 12 and thermal jacket 16 and thermal jacket 16 and lid 18 may be sealed by a plurality of o-rings or other sealing members. Thermal jacket 16 includes at least one seal 84 housed in a notch in inner face 66 proximate second end 62. Thermal jacket 16 may be configured to be secured to lid 18 to create flange seal 76 as shown in FIG. 3. Flange seal 76 creates a fluid-tight seal between thermal jacket 16 and lid 18 proximate first end 60 of cylinder 12 when flange 72 is tightened against lid 18 as shown. Flange 72 of thermal jacket 16 includes a plurality of apertures 86 configured to facilitate securing thermal jacket 16 to lid 18. Apertures 86 may be threaded to receive a bolt or set screw that secures flange 72 to lid 18 as shown in FIG. 3. Alternatively, apertures 86 may be smooth bored to allow a bolt or other fastener to pass through to secure flange 72 to lid 18.

Alternatively, in an embodiment not shown, thermal jacket 16 may include electric heating elements embedded in a thermal jacket or the electric heating elements being otherwise applied to a portion of main cylinder 12. This embodiment necessarily includes a source of electricity including, but no limited to one or a combination of batteries, a generator, or a conventional plug into the public electricity grid. The thermal jacket of this embodiment may be fabric, plastic, carbon fiber, metal or any other configuration now known or hereafter developed that facilitates heat transfer from electric heating elements to main cylinder 12. One embodiment includes thermal jacket being flexible such that thermal jacket can be wrapped around main cylinder 12. The electric heat element is preferably radiant; however, any known electric heating method now known or hereafter developed is within the scope of the present invention. In any event, a thermostat (not shown) or other temperature control device or switch as now known or hereafter developed may be in communication with the thermal jacket of the present invention and activate the thermal jacket as necessary to maintain a temperature that substantially matches the actual bottomhole temperature for that sample.

Lid 18 of HTHP reactor 10 is generally configured to be removably coupled to main cylinder 12 using any pressure resistant connection type known in the art or hereafter developed. Lid 18 is also generally disposed to allowing a technician to mount a plurality of various instruments in communication with reactor chamber 48 to observe the conditions and results of the tests. Lid 18 is generally a solid piece of material wherein the above features are milled or machined into the final piece.

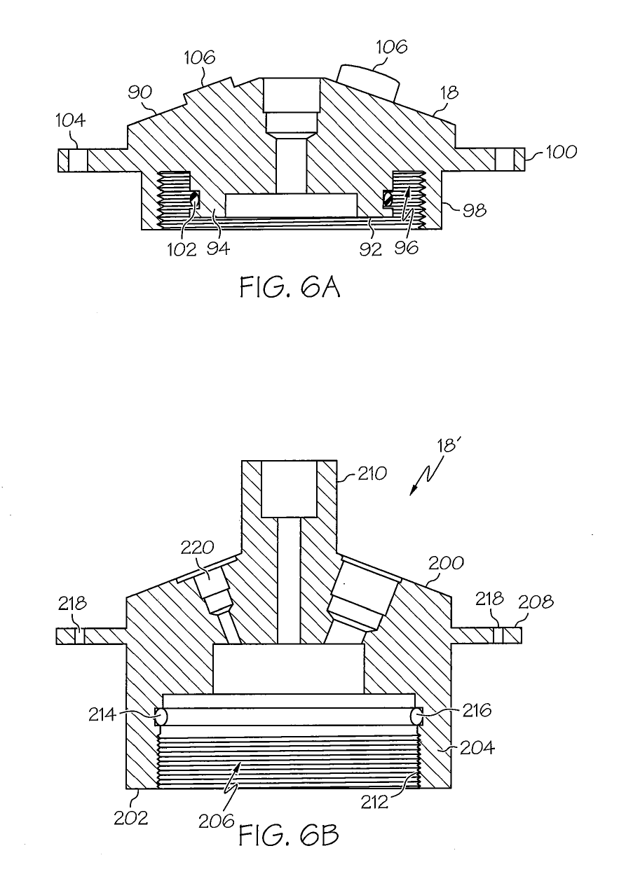

One embodiment of lid 18 shown in FIG. 6A includes a top face 90, a bottom face 92, a cylinder plug portion 94, cylinder channel 96, collar 98, and flange 100. When lid 18 is coupled to main cylinder 12, main cylinder plug portion 94 extends a distance inside cylinder 12. The cylinder plug portion 94 includes an outer diameter that is equal to or slightly less than inner diameter of main cylinder 12. Seal 102 may be included in the interface between the outer surface of the cylinder plug portion and the inner surface 26 of main cylinder 12 to prevent migration of fluid or gas out of reaction chamber 48. Cylinder channel 96 is configured to receive the walls of main cylinder 12 when lid 18 is coupled to cylinder 12 as shown in FIG. 3. In general, cylinder channel 96 may be defined by cylinder plug portion 94 on the inside and by collar 98 on the outside. A portion of collar 98 may be threaded and configured to engage a threaded first end 22 of main cylinder 12. Cylinder channel 96 generally braces open first end 22 of main cylinder 12 when first end 22 of main cylinder 12 is received into channel 96 thereby reinforcing the open end of cylinder 12 at a known weak connection point. Lid 18 twists onto main cylinder 12 via the threads 32 to secure lid 18 to cylinder 12 to create a pressure resistant connection.

An alternative embodiment shown in FIG. 6B includes lid 18′ having a top face 200, a bottom face 202, a collar 204, a cylinder housing 206, a flange 208 and a neck 210. When lid 18′ is coupled to main cylinder 12, cylinder 12 extends a distance inside cylinder housing 206. Cylinder housing 206 is defined by an inside wall 212 of collar 204. Inside wall 212 is defined by a diameter that is equal to or slightly greater than the outer diameter of main cylinder 12. A seal 214 may be included in the interface between collar 204 and main cylinder 12 as shown in FIG. 6B to prevent migration of fluid or gas out of reaction chamber 48. Seal 216 may otherwise be disposed on the interface between main cylinder 12 or, collar 204. A portion of collar 204 may be threaded and configured to engage threaded first end 22 of main cylinder 12 (shown in FIG.4). Lid 18′ generally twists onto main cylinder 12 via the threads 32 to secure lid 18′ to main cylinder 12 and compress seal 214 to create a pressure resistant connection.

Flange 100, 208 of lid 18, 18′ may also include coupling apertures 104, 218 that compliment the pattern of coupling apertures 86 through flange 72 of thermal jacket 16. The coupling apertures 86 and 104, 218 are configured to facilitate the two members 16 and 18 being temporarily secured together. The temporary coupling of the two flanges 72 and 100, 208 may be achieved using any coupling method now known or hereafter developed including set screws, bolts, and clamps.

As shown in FIGS. 6A, 6B, 7A and 7B lids 18 and 18′ also generally include a plurality of instrument housings 106, 220 configured to allow a plurality of instruments to be mounted on lid 18, 18′ and in functional communication with reaction chamber 48. Instrument housings 106, 220 are generally configured such that instruments may be mounted upon lid 18, 18′ such that the connection between lid 18, 18′ and the instruments resists the high pressures applied to reaction chamber 48. The housings 106, 220 may be of a uniform size so that a technician can alter the configuration of instruments depending on which characteristics the testing is meant to measure or determine or, alternatively, may be configured for a particular instrument or tool. The embodiment shown in FIG. 6A generally includes raised housing that are machined or coupled to lid 18. The embodiment shown in FIG. 6B generally includes top face 200 of lid 18′ being substantially planar wherein housings 220 are recessed in the body of lid 18′. FIGS. 7A and 7B illustrate an embodiment of the present invention that includes the following instruments: stirrer 110, thermowell 112, pressure gauge 114, and pH-sensor 116. However, any instruments known or hereafter developed are within the scope of the present invention, including a thermometer. Further, lid 18, 18′ may include an inlet valve 118, an outlet valve 120, and/or a pressure relief valve 119 to either add or remove contents from reaction chamber 48 as shown in FIG. 7A.

End cap 20 is generally coupled to second end 24 of main cylinder 12 providing a pressure resistant connection thereby allowing pressure to build up in pressurization chamber 46 and thereby applying pressure to reaction chamber 48 via piston 14. Now turning to FIG. 8, end cap 20 generally includes a top face 129, a bottom face 130, an inner side face 132, an outer side face 134, a side thickness 136 and an end thickness 138. The cross-sectional shape of end cap 20 generally corresponds and compliments the cross-sectional shape of main cylinder 12. As shown in FIG. 1, an embodiment of the present invention includes main cylinder 12 and end cap 20 being circular. In this embodiment inner side face 132 is defined by an inner diameter wherein said inner diameter is equal to or slightly greater than outer diameter of main cylinder 12 as shown in FIG. 3. A portion of inner side face 132 may include threads 140 wherein threads 140 are configured to engage threads 32 of main cylinder 12. End cap 20 may be removably coupled to main cylinder 12 by twisting it about threaded second end 24 of main cylinder 12 wherein the threaded connection secures end cap to main cylinder. End cap 20 may be configured to receive the connection to pressure regulation system, hydraulic, air, solenoid controls through any method now known or hereafter developed as shown in FIG. 8.

Another embodiment, illustrated in FIG. 3, includes a cylinder bottom 142 having an inner face 144, an outer face 146, a protuberance 148 extending outwardly from outer face 146, a cylinder plug section 150, and a flange 152. In this embodiment, cylinder plug section 150 is configured to extend a distance into main cylinder 12. Cylinder plug section 150 may further include a seal member 156 on inner face that engages inner face 26 of main cylinder 12. Flange 152 abuts second end 24 of main cylinder 12 and end cap 20 slides over cylinder bottom 142. End cap 20 includes an aperture configured such that protuberance 148 of cylinder bottom 142 may pass through and extend outwardly from main cylinder 12 as shown. Protuberance 148 is configured to receive the connection to a pressure regulation system as described above. End cap 20 including threaded inner side face 132 is twisted upon second end 24 of main cylinder 12 thereby sandwiching cylinder bottom 142 against main cylinder 12. The threaded connection allows cylinder bottom 142 to be tightened against main cylinder 12 by end cap 20 providing the necessary pressure resistant connection.

To construct one embodiment of the HTHP reactor 10 of the present invention, piston 14 is placed within main cylinder 12. Cylinder bottom 142 is placed adjacent to second end 24 of main cylinder 12. End cap 20 is twisted over cylinder bottom 142 about main cylinder 12 and tightened to sandwich cylinder bottom 142 between end cap 20 and main cylinder 12 such that an air tight, pressure resistant connection results. Thermal jacket 16 is slid over first end 22 of main cylinder 12 and lid 18 is twisted upon the threaded first end 22 of main cylinder 12. Thermal jacket 16 is coupled to lid 18, 18′ using fasteners through apertures 84 and 104, 218. The instrumentation desired is selected and mounted in housings 106, 220 on lid 18, 18′. HTHP reactor 10 may be assembled in various different ways and is not restricted to an assembly in a certain order or configuration.

From the foregoing it will be seen that this invention is one well adapted to attain all ends and objects hereinabove set forth together with the other advantages which are obvious and which are inherent to the structure. It will be understood that certain features and subcombinations are of utility and may be employed without reference to other features and subcombinations. This is contemplated by and is within the scope of the claims. Since many possible embodiments may be made of the invention without departing from the scope thereof, it is to be understood that all matter herein set forth or shown in the accompanying drawings is to be interpreted as illustrative, and not in a limiting sense.

What is claimed is:

1. A high temperature high pressure microbial reactor comprising:
a main cylinder having a first end and a second end;
a reversible piston moveable within said main cylinder, said piston dividing said main cylinder into a first chamber and a second chamber;
a thermal jacket surrounding at least a portion of said cylinder proximate said first chamber;
a lid removably coupled to said first end of said cylinder providing a pressure resistant connection and wherein said thermal jacket is removably coupled to said lid; and
an end cap removably coupled to said second end of said cylinder providing a pressure resistant connection;
wherein the high temperature high pressure microbial reactor is configured such that the in-situ temperature and pressure of an underground resource reservoir is substantially maintained during experiments including a bottomhole sample from said reservoir.

2. The high temperature high pressure microbial reactor of claim 1 wherein the high temperature high pressure microbial reactor is configured to substantially maintain the in-situ temperature and pressure of said bottomhole sample during the transfer of said sample from a sampler or a transport vessel to said high temperature high pressure microbial reactor.

3. The high temperature high pressure microbial reactor of claim 1 wherein said first chamber is configured to receive said sample.

4. The high temperature high pressure microbial reactor of claim 3 wherein said second chamber is configured to receive a volume of pressurized liquid or gas.

5. The high temperature high pressure microbial reactor of claim 4 wherein said volume of pressurized liquid or gas received into said second chamber compresses said piston against said sample received into said first chamber.

6. The high temperature high pressure microbial reactor of claim 4 wherein said piston is configured to prevent intermingling between said sample in said first chamber and said volume of pressurized liquid or gas in said second chamber.

7. The high temperature high pressure microbial reactor of claim 1 wherein said piston floats within said main cylinder and is configured to maintain a substantially constant pressure during changes in a volume of fluid or gas in said first chamber.

8. The high temperature high pressure microbial reactor of claim 1 wherein said end cap is configured to receive a pressurization element to introduce a pressurized volume of liquid or gas into said second chamber.

9. The high temperature high pressure microbial reactor of claim 1 further comprising a cylinder bottom member wherein said cylinder bottom member is compressed between said end cap and said second end of said main cylinder.

10. The high temperature high pressure microbial reactor of claim 9 wherein said cylinder bottom member is configured to receive a pressurization element to introduce a volume of liquid or gas into said second chamber.

11. The high temperature high pressure microbial reactor of claim 1 wherein said lid further includes a plurality of housings configured to receive one or more instrument.

12. The high temperature high pressure microbial reactor of claim 11 wherein said instrument is selected from a group consisting of a stirrer, a thermowell, a thermometer, a pressure gauge, and a pH-sensor.

13. The high temperature high pressure microbial reactor of claim 11 wherein said one or more instrument is mounted in said housing and is in functional communication with said first chamber.

14. The high temperature high pressure microbial reactor of claim 1 wherein said lid further includes a pH sensor mounted thereon, said pH sensor capable of measuring pH while subjected to high pressure.

15. A high temperature high pressure microbial reactor comprising:
a main cylinder having a first end and a second end;
a reversible piston moveable within said main cylinder, said piston dividing said main cylinder into a first chamber and a second chamber;
a thermal jacket surrounding at least a portion of said cylinder proximate said first chamber to heat or cool at least said first chamber of said main cylinder;
a lid removably coupled to said first end of said cylinder to provide high pressure resistant connection and wherein said thermal jacket is removably coupled to said lid; and
an end cap removably coupled to said second end of said cylinder;
wherein the high temperature high pressure microbial reactor is configured such that the in-situ temperature and pressure of an underground resource reservoir sample is substantially maintained during experiments including a liquid material sample from said reservoir;
wherein said first chamber is configured to receive said sample and said second chamber is configured to receive a volume of pressurized liquid or gas; and
wherein said piston is configured to prevent intermingling between said sample in said first chamber and said volume of pressurized liquid or gas in said second chamber.

16. The high temperature high pressure microbial reactor of claim 15 wherein said piston floats within said main cylinder and is configured to maintain a substantially constant pressure during changes in a volume of liquid in said first chamber.

17. The high temperature high pressure microbial reactor of claim 15 wherein said lid further includes a pH sensor mounted thereon, said pH sensor capable of measuring pH while subjected to high pressure.

18. A high temperature high pressure microbial reactor for testing microbes contained in an underground oil reservoir comprising:
a main cylinder having a first end and a second end;
a reversible piston moveable within said main cylinder, said piston dividing said main cylinder into a first chamber proximate said first end and a second chamber proximate said second end wherein said first chamber is configured to receive a fluid sample from the underground oil reservoir and said second chamber is configured to receive a volume of pressurized liquid or gas and wherein said piston is configured to prevent intermingling between said sample in said first chamber and said volume of pressurized liquid or gas in said second chamber;
a thermal jacket surrounding at least a portion of said cylinder proximate said first chamber wherein said thermal jacket transfers heat to said main cylinder to maintain a temperature substantially equal to the temperature of the underground oil reservoir in said first chamber of said main cylinder;
a lid removably coupled to said first end of said cylinder to provide high pressure resistant connection and wherein said thermal jacket is removably coupled to said lid; and
an end cap removably coupled to said second end of said cylinder; and
wherein said piston floats within said main cylinder and said piston compresses against said sample in said first chamber when said volume of pressurized liquid or gas is received into said second chamber and wherein said piston is configured to maintain a substantially constant pressure during changes in a volume of the liquid in said first chamber.

19. The high temperature high pressure microbal reactor of claim 18 wherein said fluid sample has been maintained at reservoir temperature and pressure throughout sampling, transfer to a transport container and inoculation.

20. The high temperature high pressure microbial reactor of claim 1 wherein said lid further includes a pH sensor mounted thereon, said pH sensor capable of measuring pH while subjected to high pressure.
